(19) United States
(12) Patent Application Publication
(10) Pub. No.: US 2026/0088125 A1
(43) Pub. Date: Mar. 26, 2026

(54) DESIGN OF SELECTIVELY TRANSLATED mRNAS

(71) Applicant: RAMOT AT TEL-AVIV UNIVERSITY LTD., Tel Aviv (IL)

(72) Inventors: Tamir TULLER, Tel Aviv (IL); Matan ARBEL, Tel Aviv (IL); Yehuda LANDAU, Ramat Gan (IL); Efi MOREE, Tel Aviv (IL)

(21) Appl. No.: 19/108,578

(22) PCT Filed: Sep. 5, 2023
(86) PCT No.: PCT/IL2023/050954

§ 371 Date: Mar. 4, 2025

Related U.S. Application Data
(60) Provisional application No. 63/403,813, filed on Sep. 5, 2022.

(51) Int. Cl. G16B 25/10; C12N 15/67; G16B 15/10; G16B 40/20

(52) U.S. Cl. CPC G16B 25/10; C12N 15/67; G16B 15/10; G16B 40/20

(57) ABSTRACT

Methods of designing a synthetic RNA molecule comprising obtaining a coding sequence encoding a protein of interest; obtaining a sequence of an RNA expressed in a first tissue or cell type and not expressed or lowly expressed in a second tissue or cell type; selecting a region within the sequence of an RNA; and producing a sequence of a synthetic RNA molecule comprising from 5′ to 3′: a reverse complement to the selected region comprising a 5′ unhybridized region and a 3′ hybridized region, a loop region that does not hybridize; a region reverse complementary to 3′ hybridized region and the obtained coding sequence encoding a protein of interest are provided. Synthetic RNA molecules produced by a method of the invention and computer program products for perform a method of the invention are also provided.

### CROSS REFERENCE TO RELATED APPLICATIONS

This application claims the benefit of priority of U.S. Provisional Patent Application No. 63/403,813, filed Sep. 5, 2022, the contents of which are all incorporated herein by reference in their entirety.

### REFERENCE TO AN ELECTRONIC SEQUENCE LISTING

The contents of the electronic sequence listing (RMT-P-026-PCT.xml; Size: 13,220 bytes; and Date of Creation: Sep. 5, 2023) is herein incorporated by reference in its entirety.

### FIELD OF INVENTION

The present invention is in the field of mRNA translational regulation.

### BACKGROUND OF THE INVENTION

The use of RNA as a therapeutic tool is a new frontier in the broad view of disease treatment and prevention. RNA-based therapies hold the potential to revolutionize fields such as vaccines, personalized medicine and cancer therapy. Since RNA is easy to manufacture, safe, modular, cost effective and holds the ability to target previously untreatable pathologies, RNA-based treatments could serve as the future standard in medicine.

One of the big challenges faced by such treatments is a lack of specificity. Current delivery methods are general, where the RNA molecules are delivered with nonspecific vehicles and arrive not only to the target cells but many more. In that case, the protein of interest is expressed in unwanted cells, which can result in high toxicity.

In recent years, various strategies were developed for control over mRNA translation, most are based on designing a switch module for sensing and responding to a specific endogenous RNA trigger that appears mainly in the target cell (e.g., the cancer cell). Among others, a toehold switch is based on the interplay between the self-folding of the mRNA and its hybridization with the trigger RNA. However, one of the main limitations for these toehold switches has been their dynamic range. Work done in prokaryotes showed that rational design of the RNA sequence can yield switches with over 400-fold mean dynamic range. Minimal work has been done with toeholds in eukaryotes and the dynamic range remains the limiting factor, as a maximum of 2-fold dynamic ranged was achieved with toeholds that are based on miRNA as their triggers. A new method, and computational model, for toehold switch design that increases the dynamic range in eukaryotes, making them applicable in RNA therapeutics is therefore greatly needed.

### SUMMARY OF THE INVENTION

The present invention provides methods of designing a synthetic RNA molecule. Synthetic RNA molecules produced by the method of the invention are also provided. Computer program products for performing a method of the invention are also provided.

According to a first aspect, there is provided a method of designing a synthetic RNA molecule, the method comprising:
a. obtaining a coding sequence encoding a protein of interest;
b. obtaining a sequence of an RNA expressed in a first tissue or cell type and not expressed or lowly expressed in a second tissue or cell type;
c. selecting a region within the sequence of an RNA; and
d. producing a sequence of a synthetic RNA molecule comprising from 5′ to 3′:
   i. a reverse complement to the selected region comprising a 5′ unhybridized region and a 3′ hybridized region;
   ii. a loop region that does not hybridize to any of (i), (ii) and (ii);
   iii. a region reverse complementary to the 3′ hybridized region of (i); and
   iv. the obtained coding sequence encoding a protein of interest;
   wherein a ribosome binding site (RBS) is located within the 3′ hybridized region of (i), within (ii) or within (iii);
thereby designing a synthetic RNA molecule.

According to another aspect, there is provided a method of designing a synthetic RNA molecule, the method comprising:
a. obtaining a coding sequence encoding a protein of interest;
b. obtaining a sequence of an RNA expressed in a first tissue or cell type and not expressed or lowly expressed in a second tissue or cell type;
c. selecting a region within the sequence of an RNA; and
d. producing a sequence of a synthetic RNA molecule comprising from 3′ to 5′:
   i. the obtained coding sequence encoding a protein of interest;
   ii. a reverse complement to the selected region comprising a 3′ unhybridized region and a 5′ hybridized region;
   iii. a loop region that does not hybridize to any of (ii), (iii) and (iv); and
   iv. a region reverse complementary to the 5′ hybridized region of (ii);
   wherein a ribosome binding site (RBS) is located within the 5′ hybridized region of (ii), within (iii) or within (iv);
thereby designing a synthetic RNA molecule.

According to some embodiments, the synthetic RNA molecule produces the protein of interest in the first tissue or cell type and does not produce or lowly produces the protein of interest in the second tissue or cell type.

According to some embodiments, the coding sequence encoding a protein of interest is codon optimized for expression in the first tissue or cell type.

According to some embodiments, the RNA expressed in a first tissue or cell type and not expressed or lowly expressed in a second tissue or cell type is not a microRNA (miRNA).

According to some embodiments, the RNA is a messenger RNA (mRNA).

According to some embodiments, the obtaining a sequence of an RNA comprises performing a statistical search algorithm on RNA expression data from the first tissue or cell type and the second tissue or cell type and ranking molecules based on their ability to distinguish the first tissue or cell type and the second tissue or cell type.

According to some embodiments, the method comprises selecting an RNA whose expression in the second tissue or cell type is sufficiently low as to not induce expression of the protein of interest in the presence of the synthetic RNA molecule and whose expression in the first tissue or cell type is sufficiently high as to induce expression of the protein of interest in the presence of the synthetic RNA molecule.

According to some embodiments, the method comprises selecting an RNA whose GC content is below a predetermined threshold.

According to some embodiments, selecting a region comprises selecting an unfolded region.

According to some embodiments, an unfolded region is a region with a local folding energy above a predetermined threshold.

According to some embodiments, the folding is determined for the sequence of an RNA.

According to some embodiments, selecting an unfolded region comprises performing a window search algorithm that produces a folding matrix indicating the probability of each nucleotide of the sequence of an RNA hybridizing to another nucleotide of the sequence of an RNA and selecting a window predicted to not hybridize.

According to some embodiments, another nucleotide of the sequence comprises the sequence of the gene of interest.

According to some embodiments, selecting a region comprises evaluating the local folding energy of windows adjacent to the region and/or the folding of the region to windows adjacent to the region.

According to some embodiments, the region is between 20 and 100 nucleotides.

According to some embodiments, the window is an outer window and wherein each outer window is subdivided into inner windows consisting of a portion that is less than 100% of the outer window and wherein the producing a folding matrix is producing a folding matrix for each inner window.

According to some embodiments, the selecting an unfolded region further comprises summing the folding energies for each inner window across all outer windows to produce an inner window folding value, summing the inner window folding values of all inner windows in each outer window to produce an outer window folding value and selecting an outer window with a folding value beyond a predetermined threshold.

According to some embodiments, the method further comprises confirming formation of a stem-loop structure comprising the 3′ hybridized region, the loop region and the region reverse complementary to the 3′ hybridized region.

According to some embodiments, the method further comprises confirming formation of a stem-loop structure comprising the 5′ hybridized region, the loop region and the region reverse complementary to the 5′ hybridized region.

According to some embodiments, the confirming comprises performing a sliding window RNA folding prediction algorithm across the full sequence of the synthetic RNA and selecting the most probable secondary structure.

According to some embodiments, the method further comprises optimizing the location of the RBS to prevent translation in the absence of the RNA expressed in a first tissue or cell type and not expressed or lowly expressed in a second tissue or cell type.

According to some embodiments, the RBS is a Kozak sequence.

According to some embodiments, the RBS is located in the loop region.

According to some embodiments, a start codon is located in the loop region and is 3′ to the RBS.

According to some embodiments, the synthetic RNA sequence between the start codon and the obtained coding sequence encoding a protein of interest is codon optimized to match a codon usage of the coding sequence encoding a protein of interest.

According to some embodiments, the codon optimization comprises codon usage bias (CUB) optimization or typical decoding rate (TDR) optimization.

According to some embodiments, a start codon of the coding sequence encoding a protein of interest is separated from the rest of the coding sequence by a linker.

According to some embodiments, the start codon is downstream of the RBS and the linker is within the region reverse complementary to the 3′ hybridized region.

According to some embodiments, the start codon is downstream of the RBS and the linker comprises the 5′ hybridized region.

According to some embodiments, the method comprises optimizing the linker to be as short as possible while expression of the protein of interest is still inhibited in the absence of the RNA expressed in a first tissue or cell type and not expressed or lowly expressed in a second tissue or cell type.

According to some embodiments, the method comprises codon optimizing the linker to match a codon usage of the coding sequence encoding a protein of interest.

According to some embodiments, the method comprises an unhybridized region between a first and second part of the 3′ hybridized region, an unhybridized region between a first and second part of the region reverse complementary to the 3′ hybridized region or both.

According to some embodiments, the method comprises an unhybridized region between a first and second part of the 5′ hybridized region, an unhybridized region between a first and second part of the region reverse complementary to said 5′ hybridized region or both.

According to some embodiments, the start codon is located in the unhybridized region between a first and second part of the region reverse complementary to the 3′ hybridized region.

According to some embodiments, the linker comprises the second part of the region reverse complementary to the 3′ hybridized region which is 3′ to the start codon.

According to some embodiments, the method further comprises confirming the synthetic RNA molecule produces the protein of interest in the first tissue or cell type and does not produce or lowly produces the protein of interest in the second tissue or cell type.

According to some embodiments, the method comprises applying a trained regressor algorithm to predict protein of interest production.

According to another aspect, there is provided a synthetic RNA molecule produced by a method of the invention.

By another aspect there is provided, a computer program product comprising a non-transitory computer-readable storage medium having program code embodied thereon, the program code executable by at least one hardware processor to perform a method of the invention.

According to another aspect, there is provided a method of producing expression of a protein of interest in a first eukaryotic tissue or cell type, the method comprising introducing the synthetic RNA molecule of the invention into the first eukaryotic tissue or cell type, thereby producing expression of a protein of interest in a first eukaryotic tissue or cell type.

According to some embodiments, the introducing further comprises introducing the synthetic RNA molecule of the invention into a second eukaryotic tissue or cell type and wherein the introducing does not produce or lowly produces expression of the protein of interest in the second eukaryotic tissue or cell type.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Schematic of a toehold switch shifting from an inactive to an active state.

FIG. 2: Block diagram of one embodiment of the trigger selection method of the invention.

FIG. 3: Schematic illustration of the window search algorithm of the invention.

FIG. 4: Block diagram of one embodiment of the toehold design method of the invention.

FIG. 5: Schematic illustration of a toehold switch structure of the invention comprising the Kozak and AUG in the loop region.

FIG. 6: Schematic illustration of a toehold switch structure of the invention comprising the trigger binding site in the 3′ side of the toehold.

FIG. 7: Bar graph of GFP expression in yeast cells containing various combinations of toehold molecules (SEQ ID NO: 1-4) and codon optimized mCherry. GFP levels are normalized to florescence levels measured in a WT yeast containing no GFP and no mCherry. The presence of trigger in each train is represented by a plus. The presence of toehold is represented by the number of the toehold molecule used. Cells without additional molecules are used to set the GFP baseline and cells expressing an open GFP plasmid are used as a positive control.

FIG. 8: Schematic illustration of a toehold switch structure of the invention comprising the Kozak and AUG 3′ to the stem-loop structure.

### DETAILED DESCRIPTION OF THE INVENTION

The present invention, in some embodiments, provides methods of designing synthetic RNA molecules that express a protein of interest in a first tissue or cell type and don't express or lowly express the protein of interest in a second tissue or cell type. The present invention further concerns synthetic RNA molecules produced by a method of the invention and computer program products for performing a method of the invention.

By a first aspect, there is provided a method designing an RNA molecule, the method comprising:
a. obtaining a coding sequence;
b. obtaining a sequence of an RNA expressed in a first tissue or cell type and lowly expressed in a second tissue or cell type; and
c. producing a sequence of an RNA molecule comprising:
   i. a reverse complement to the obtained RNA comprising a 5′ unhybridized region and a 3′ hybridized region;
   ii. a region reverse complementary to the 3′ hybridized region of (i); and
   iii. the obtained coding sequence;
thereby designing a synthetic RNA molecule.

By another aspect, there is provided a method designing an RNA molecule, the method comprising:
a. obtaining a coding sequence;
b. obtaining a sequence of an RNA expressed in a first tissue or cell type and lowly expressed in a second tissue or cell type; and
c. producing a sequence of an RNA molecule comprising:
   i. a reverse complement to the obtained RNA comprising a 3′ unhybridized region and a 5′ hybridized region;
   ii. a region reverse complementary to the 5′ hybridized region of (i); and
   iii. the obtained coding sequence;
thereby designing a synthetic RNA molecule.

In some embodiments, the method is an in vitro method. In some embodiments, the method is an in silico method. In some embodiments, the method is an ex vivo method. In some embodiments, the method is a computerized method. In some embodiments, the method cannot be performed in a human mind. In some embodiments, the method is a method of producing a synthetic RNA molecule. In some embodiments, the method further comprises producing the synthetic RNA molecule from the designed sequence. In some embodiments, producing the molecule is synthesizing the molecule. In some embodiments, the designed sequence is the produced sequence. Methods of nucleic acid molecule synthesis are well known in the art and are available commercially. Any such method may be used to synthesize the synthetic RNA of the invention.

In some embodiments, the RNA molecule is a synthetic RNA molecule. In some embodiments, the RNA molecule is an engineered RNA molecule. In some embodiments, the RNA molecule is a chimeric RNA molecule. In some embodiments, the RNA molecule is a toehold molecule. In some embodiments, the RNA molecule comprises a toehold. In some embodiments, the RNA molecule is translated in the first tissue or cell type. In some embodiments, the RNA molecule produces protein in the first tissue or cell type. In some embodiments, translated is highly translated. In some embodiments, producing protein is highly producing protein. In some embodiments, the RNA molecule is lowly translated in the second tissue or cell type. In some embodiment, lowly translated is untranslated. In some embodiments, the RNA molecule lowly produces protein in the second tissue or cell type. In some embodiments, lowly produces is does not produce.

In some embodiments, the first tissue or cell type is a eukaryotic tissue or cell type. In some embodiments, the first cell type is a eukaryotic cell. In some embodiments, the second tissue or cell type is a eukaryotic tissue or cell type. In some embodiments, the second cell type is a eukaryotic cell. In some embodiments, a eukaryotic cell is a yeast cell. In some embodiments, a eukaryotic cell is selected from a yeast cell and a mammalian cell.

In some embodiments, highly is above a predetermined threshold. In some embodiments, highly is higher than in the second tissue or cell type. In some embodiments, highly is detectably. In some embodiments, lowly is below a predetermined threshold. In some embodiments, lowly is lower than in the first tissue or cell type. In some embodiments, lowly is undetectably. In some embodiments, highly is to produce a functional amount. In some embodiments, lowly is to produce an amount of protein that is not functional.

In some embodiments, the protein is at least 5, 10, 20, 30, 40, 50, 60, 70, 80, 90, 100, 150, 200, 250, 300, 400, 500, 600, 700, 800, 900 or 1000 times more greatly produced in the first tissue or cell type than the second tissue or cell type. Each possibility represents a separate embodiment of the invention. In some embodiments, the protein is at least 10 times more greatly produced in the first tissue or cell type than the second tissue or cell type. In some embodiments, the dynamic range of expression of the protein between the two tissues is greater than 2, 3, 4, 5, 6, 7, 8, 9, 10, 20, 30, 40, 50, 60, 70, 80, 90, 100, 150, 200, 250, 300, 400, 500, 600, 700, 800, 900 or 1000. Each possibility represents a separate embodiment of the invention. In some embodiments, the dynamic range of expression of the protein between the two tissues is greater than 2. In some embodiments, the dynamic range of expression of the protein between the two tissues is greater than 10.

In some embodiments, the coding sequence encodes the protein. In some embodiments, the protein is a protein of interest. In some embodiments, the protein of interest is a therapeutic protein. In some embodiments, the protein of interest is a toxic protein. In some embodiments, the therapeutic protein is an anticancer protein. In some embodiments, the protein of interest treats a disease. In some embodiments, the therapeutic protein is a vaccine. In some embodiments, the therapeutic protein is a protein of a pathogen. In some embodiments, the protein of interest is a protein to be expressed in the first tissue or cell type and not expressed in the second tissue or cell type. In some embodiments, the protein of interest is a protein to be preferentially expressed in the first tissue or cell type over the second tissue or cell type.

In some embodiments, the coding sequence is codon optimized for expression in a target organism. In some embodiments, the organism is a mammal. In some embodiments, the mammal is a human. In some embodiments, the organism comprises both the first and second tissues or cell types. In some embodiments, the coding sequence is codon optimized for expression in the first tissue or cell type. In some embodiments, the coding sequence is codon deoptimized for expression in the second tissue or cell type.

In some embodiments, the codon bias is optimized. In some embodiments, calculating codon usage comprises calculating codon usage bias (CUB). In some embodiments, codon bias is optimized to match the codon bias in the first organism. In some embodiments, codon bias is optimized to not match the codon bias in the second organism. In some embodiments, codon optimized comprises codon usage bias (CUB) optimization. In some embodiments, the CUB is codon bias. In some embodiments, CUB optimization comprises tRNA adaptation index (tAI) optimization. In some embodiments, CUB optimization is by tAI. In some embodiments, CUB optimization comprises codon adaptation index (CAI) optimization. In some embodiments, CUB optimization is by CAI. In some embodiments, CUB optimization comprises typical decoding rate (TDR) optimization. In some embodiments, CUB optimization is by TDR. Performance of CUB, tAI, CAI, TDR and other algorithmic optimizations are well known in the art and are further described hereinbelow. A skilled artisan with a target organism, tissue or cell type, coding sequences of genes expressed in the target organism/tissue/cell type and expression levels of those sequences in the target organism/tissue/cell type can calculate the indexes and biases recited herein. Thus, optimization may include replacing a given codon in the codon region by a synonymous but more frequently used codon in the first tissue/cell type (or organism) or a synonymous but less frequently used codon in the second tissue/cell type. In some embodiments, the frequency is calculated by tAI. In some embodiments, the frequency is calculated by CAI. In some embodiments, the frequency is calculated by TDR. In some embodiments, calculation is relative to null model. In some embodiments, the null model is a VCUB null model. Methods of generating and analyzing these null models are well known in the art.

In some embodiments, expressed is highly expressed. In some embodiments, expressed is detectably expressed. In some embodiments, expressed is expressed above a predetermined threshold. In some embodiments, the threshold is the concentration sufficient to induce translation of the RNA of the invention. In some embodiments, lowly expressed is unexpressed. In some embodiments, lowly expressed is undetectably expressed. In some embodiments, lowly expressed is expressed below a predetermined threshold.

In some embodiments, an RNA expressed in a first tissue or cell type and lowly expressed in a second tissue or cell type is a trigger RNA. In some embodiments, the trigger RNA is a messenger RNA (mRNA). In some embodiments, the trigger RNA is not a microRNA (miRNA). In some embodiments, the trigger RNA comprises a coding region.

In some embodiments, obtaining a sequence of an RNA comprises performing a search algorithm. In some embodiments, the algorithm is a computerized algorithm. In some embodiments, the search algorithm is a statistical search algorithm. In some embodiments, the search algorithm is performed on RNA expression data from the first tissue or cell type and the second tissue or cell type. In some embodiments, the search algorithm ranks RNA molecules based on their ability to distinguish the first tissue or cell type and the second tissue or cell type.

In some embodiments, obtaining a sequence comprises selecting an RNA whose expression in the second tissue or cell type is sufficiently low as to not induce expression of the protein in the presence of the RNA molecule of the invention. In some embodiments, expression is protein expression. In some embodiments, obtaining a sequence comprises selecting an RNA whose expression in the first tissue or cell type is sufficiently high as to induce expression of the protein in the presence of the RNA molecule of the invention.

In some embodiments, obtaining a sequence comprises selecting an RNA comprising a GC content below a predetermined threshold. In some embodiments, obtaining a sequence comprises selecting an RNA comprising as low a GC content as possible. In some embodiments, obtaining a sequence comprises selecting an RNA comprising RNA folding below a predetermined threshold. In some embodiments, RNA folding is local RNA folding. In some embodiments, obtaining a sequence comprises selecting an RNA comprising as little RNA folding as possible.

In some embodiments, the method further comprises selecting a region within the sequence of the RNA. In some embodiments, the method further comprises selecting a region within the sequence of the trigger RNA. In some embodiments, the region is an unfolded region. In some embodiments, an unfolded region is a region with a folding energy above a predetermined threshold. In some embodiments, the region is the most unfolded region in the RNA. In some embodiments, unfolded comprises the highest local folding energy. In some embodiments, the highest local folding energy is the less negative local folding energy. In some embodiments, local is within a window of nucleotides. In some embodiments, the window is the region. In some embodiments, the folding is determined for the sequence of an RNA. In some embodiments, the folding is determined for the region. In some embodiments, the folding is determined for windows throughout the sequence of an RNA. In some embodiments, the folding is determined for windows throughout the region.

In some embodiments, determining local folding energy comprises inputting the sequence into a folding program. In some embodiments, a folding program is a program that predicts RNA folding. In some embodiments, a folding program is a program that models RNA folding. In some embodiments, a folding program provides a folding energy for a sequence. In some embodiments, the folding energy is local folding energy. In some embodiments, local is over a given window. In some embodiments, the window is about 40 nucleotides (nt). In some embodiments, the window is about 60 nt. In some embodiments, the window is 20-150 nt. In some embodiments, the window is 20-100 nt. In some embodiments, the window is 20-60 nt. In some embodiments, the window is 20-50 nt. In some embodiments, the window is 20-40 nt. In some embodiments, the window is 36-40 nt. In some embodiments, the window is 30-40 nt. In some embodiments, the window is 30-36 nt. In some embodiments, the window is 40-150 nt. In some embodiments, the window is 50-150 nt. In some embodiments, the window is 60-150 nt. In some embodiments, the window is 40-120 nt. In some embodiments, the window is 50-120 nt. In some embodiments, the window is 60-120 nt. In some embodiments, the window is 40-100 nt. In some embodiments, the window is 50-100 nt. In some embodiments, the window is 60-100 nt. In some embodiments, the window is the region. Examples of folding programs are well known in the art and include for example, Mfold, RNAfold, RNA123, RNAshapes, RNAstructure, RNAstructureWeb, RNAslider and UNAFold to name but a few. In some embodiments, local folding energy is determined with RNAfold.

In some embodiments, selecting an unfolded region comprises performing a search algorithm. In some embodiments, the search algorithm is a window search algorithm. In some embodiments, the algorithm produces a folding matrix. In some embodiments, the folding matrix indicates the probability that each nucleotide hybridizes to another nucleotide. In some embodiments, each nucleotide is each nucleotide of the sequence of the RNA. In some embodiments, each nucleotide is each nucleotide of the window. In some embodiments, each nucleotide is each nucleotide of the region. In some embodiments, the another nucleotide is within the sequence of the RNA. In some embodiments, the another nucleotide is within the region. In some embodiments, the another nucleotide is within the window. In some embodiments, the another nucleotide is within the sequence of the gene of interest. In some embodiments, the another nucleotide is every other nucleotide in the molecule. In some embodiments, the molecule includes the sequence of the gene of interest. In some embodiments, the selecting an unfolding region further comprises selecting a window predicted to not hybridize. In some embodiments, the prediction is based on the probability matrix. In some embodiments, a window predicted to not hybridize is the window with the lowest probability to hybridize.

In some embodiments, selecting a region further comprises evaluating the local folding energy of a window adjacent to the region. In some embodiments, a window adjacent is both windows adjacent to the region. In some embodiments, evaluating the folding is determining local folding in the adjacent windows. In some embodiments, evaluating the folding is determining folding to the adjacent windows. In some embodiments, evaluating the folding is determining the folding from the region to the windows adjacent to the region.

In some embodiments, the window is an outer window. In some embodiments, an outer window is subdivided into inner windows. In some embodiments, the outer window comprises a plurality of inner windows. In some embodiments, an inner window is a portion of the outer window. In some embodiments, a portion is less than 100 percent. In some embodiments, an inner window is between 5-20, 6-20, 7-20, 8-20, 9-20, 10-20, 15-20, 5-18, 6-18, 7-18, 8-18, 9-18, 10-18, 15-18, 5-15, 6-15, 7-15, 8-15, 9-15, 10-15, 5-12, 6-12, 7-12, 8-12, 9-12, 10-12, 5-10, 6-10, 7-10, 8-10, or 9-10 nucleotides in length. Each possibility represents a separate embodiment of the invention. In some embodiments, each window is 1 nucleotide shifted from another window. In some embodiments, the inner windows are produced by starting at an end of the outer window and producing a window, shifting over 1 nucleotide and producing another window and repeating and until the end of the outer window. In some embodiments, a folding matrix is produced for each inner window. In some embodiments, a folding energy is produced for each inner window. In some embodiments, a folding value is produced for each inner window. In some embodiments, a folding value is a folding score. In some embodiments, the folding energies for each inner window across all outer windows is summed. In some embodiments, the summing produces an inner window folding value. In some embodiments, a value is a score. In some embodiments, the inner window folding values for all inner windows in an outer window are summed. In some embodiments, the summing produces an outer window folding value. In some embodiments, the selecting comprises selecting an outer window. In some embodiments, an outer window with a folding value beyond a predetermined threshold is selected. In some embodiments, beyond is above. In some embodiments, beyond is below.

In some embodiments, the RNA molecule comprises a reverse complement to the sequence of an RNA. In some embodiments, the RNA molecule comprises a reverse complement to a selected region. In some embodiments, the reverse complement comprises two regions. In some embodiments, the two regions are an unhybridized region and a hybridized region. In some embodiments, the unhybridized region is 5′ to the hybridized region. In some embodiments, the unhybridized region is 3′ to the hybridized region. In some embodiments, the unhybridized region is a 5′ region. In some embodiments, the unhybridized region is a 3′ region. In some embodiments, the region reverse complementary comprises at least 1, 2, 3, 4, 5, 6, 7, 8, 9, 10, 12, 15, 17, 20, 25, 30, 35, 40, 45, or 50 nucleotides. Each possibility represents a separate embodiment of the invention. In some embodiments, the region reverse complementary comprises at most 10, 12, 15, 17, 20, 25, 30, 35, 40, 45, 50, 60, 70, 75, 80, 90 or 100 nucleotides. Each possibility represents a separate embodiment of the invention. In some embodiments, the region reverse complementary and the 3′ hybridized region are the same size. In some embodiments, the region reverse complementary and the 5′ hybridized region are the same size. In some embodiments, the reverse complement and the selected region are the same size.

In some embodiments, the RNA molecule further comprises a loop region. In some embodiments, the loop region does not hybridize to itself. In some embodiments, the loop region does not hybridize to the reverse complement of the selected region. In some embodiments, the loop region does not hybridize to the region reverse complementary to the hybridized region. In some embodiments, the loop region does not hybridize to any sequence in the RNA of the invention. In some embodiments, the loop region does not hybridize to any sequence. In some embodiments, the loop is not a bulge. In some embodiments, the loop comprises or consists of at least 1, 2, 3, 4, 5, 6, 7, 8, 9, 10, 12, 15, 17, 20, 25, 30, 35, 40, 45, or 50 nucleotides. Each possibility represents a separate embodiment of the invention. In some embodiments, the loop comprises or consists of at most 10, 12, 15, 17, 20, 25, 30, 35, 40, 45, 50, 60, 70, 75, 80, 90 or 100 nucleotides. Each possibility represents a separate embodiment of the invention. In some embodiments, a loop comprises or consists of at least 5 nucleotides. In some embodiments, a loop comprises or consists of at least 8 nucleotides. In some embodiments, a bulge comprises or consists of less than 5 nucleotides. In some embodiments, a bulge comprises or consists of less than 8 nucleotides.

In some embodiments, the RNA of the invention comprises a ribosome binding site (RBS). In some embodiments, the RBS is located in the 3′ hybridized region. In some embodiments, the RBS is located in the 3′ hybridized region of the reverse complement to the selected region. In some embodiments, the RBS is located in the loop region. In some embodiments, the RBS is located in the region reverse complementary to the hybridized region. In some embodiments, the RBS is located in the region reverse complementary to the 3′ hybridized region. In some embodiments, the RBS is located 3′ to the toehold structure. In some embodiments, the RBS is located 3′ to the stem-loop structure. In some embodiments, the RBS is located 3′ to the region reverse complementary to the 3′ hybridized region. In some embodiments, the RBS is located 3′ to the 3′ unhybridized region. In some embodiments, the RBS is located between the region reverse complementary to the 3′ hybridized region and the coding sequence encoding a protein of interest. In some embodiments, the RBS is located between the coding sequence encoding a protein of interest and the reverse complement to the selected region. In some embodiments, the RBS is located between the coding sequence encoding a protein of interest and the 3′ unhybridized region.

In some embodiments, the RBS is a Kozak sequence. In some embodiments, the Kozak sequence comprises the start codon. In some embodiments, the Kozak sequence is ACC. In some embodiments, the Kozak sequence is or comprises AGAAACCAAA. In some embodiments, the Kozak sequence is or comprises AAACCAAA. In some embodiments, the Kozak sequence is or comprises ACCAAA. In some embodiments, the RBS is 5′ to the start codon. In some embodiments, the start codon is 3′ to the RBS. In some embodiments, the Kozak is ACCAAAATG. In some embodiments, the start codon is 3′ to the RBS. In some embodiments, the Kozak comprises ACCAAAATG. Improved AUG context in eukaryotes was calculated based on models described in Zur and Tuller, 2013, “New Universal Rules of Eukaryotic Translation Initiation Fidelity”, PLoS Comput. Biol. 2013; 9(7):e1003136, herein incorporated by reference in its entirety. The improved AUG context produced a superior Kozak to the canonical sequence of ACCATG.

In some embodiments, the method further comprises confirming formation of a stem loop structure. In some embodiments, the stem comprises the 3′ hybridized region hybridized to the region reverse complementary to the 3′ hybridized region. In some embodiments, the stem comprises the 5′ hybridized region hybridized to the region reverse complementary to the 5′ hybridized region. In some embodiments, the stem comprises a region of the reverse complement to the selected region hybridized to the region reverse complementary to the 3′ hybridized region. In some embodiments, the stem comprises a region of the reverse complement to the selected region hybridized to the region reverse complementary to the 5′ hybridized region. In some embodiments, the stem-loop structure comprises the 3′ hybridized region of the reverse complement to the selected region, the loop region and the region reverse complementary to the 3′ hybridized region. In some embodiments, the stem-loop structure comprises the 5′ hybridized region of the reverse complement to the selected region, the loop region and the region reverse complementary to the 5′ hybridized region.

In some embodiments, the confirming comprises performing a folding prediction algorithm. In some embodiments, folding prediction algorithm is an RNA folding prediction algorithm. In some embodiments, the folding prediction algorithm is a sliding window folding prediction algorithm. In some embodiments, the algorithm is performed across a full sequence of the RNA of the invention. In some embodiments, the algorithm is performed across a full sequence of the RNA of the invention excluding the coding region. In some embodiments, the algorithm is performed across a full sequence of the RNA of the invention excluding the coding region encoding the protein of interest. In some embodiments, the most probable secondary structure is selected. In some embodiments, the confirming further comprises selecting the most probable secondary structure.

In some embodiments, the method further comprises optimizing the location of the RBS. In some embodiments, the optimizing is optimizing prevention of translation in the absence of the trigger RNA. It will be understood by a skilled artisan that the RBS regulates the binding of the ribosome. The position of the RBS thus has a strong effect on translation. Therefore, the RBS will be positioned such that there is no, or minimal leakage of translation in the second tissue or cell type. That is in the absence of the trigger RNA there is minimal or no ribosome binding and/or translation. In some embodiments, the optimal RBS position is within the loop region.

In some embodiments, the start codon is located 3′ to RBS. In some embodiments, the start codon is AUG. In some embodiments, the AUG is located in the loop. In some embodiments, the start codon is 1-10 nucleotides downstream of the RBS. In some embodiments, the start codon is 6-8 nucleotides downstream of the RBS. In some embodiments, the start codon is 3 nucleotides downstream of the RBS. In some embodiments, the start codon is 4 nucleotides downstream of the RBS. In some embodiments, the RBS is a Kozak sequence and the Kozak sequence comprises the start codon. In some embodiments, the position of the start codon relative to the RBS is optimized. In some embodiments, the start codon is located in the 3′ hybridized region. In some embodiments, the start codon is located in the 3′ hybridized region of the reverse complement to the selected region. In some embodiments, the start codon is located in the loop region. In some embodiments, the start codon is located in the region reverse complementary to the hybridized region. In some embodiments, the start codon is located in the region reverse complementary to the 3′ hybridized region.

In some embodiments, the start codon is discontinuous with the rest of the coding region. In some embodiments, the start codon of the coding sequence is separated from the rest of the coding sequence. In some embodiments, a linker is inserted between the start codon and the rest of the coding region. In some embodiments, the start codon is separated from the rest of the coding sequence with a linker. In some embodiments, the linker is within the region reverse complementary to the 3′ hybridized region. In some embodiments, the linker comprises the 5′ hybridized region. In some embodiments, the linker is within the 5′ hybridized region. In some embodiments, the linker comprises the region reverse complementary to the 3′ hybridized region. In some embodiments, the linker is in the loop region 3′ to the start codon. In some embodiments, the linker comprises a part of the loop region 3′ to the start codon. In some embodiments, the linker consists of the loop region 3′ to the start codon and the region reverse complementary to the 3′ hybridized region. In some embodiments, the linker consists of the region from the start codon to the rest of the coding sequence.

In some embodiments, the RNA sequence between the start codon and the obtained coding sequence is optimized. In some embodiments, the RNA sequence between the start codon and the rest of the coding sequence is optimized. In some embodiments, the linker is optimized. In some embodiments, optimized is codon optimized. In some embodiments, codon optimized is optimized to match the coding sequence. In some embodiments, the codon optimized is codon usage optimized. In some embodiments, codon optimized is CUB optimized. In some embodiments, codon optimized is TDR optimized. In some embodiments, optimized is length optimized. In some embodiments, optimal length is as short as possible. In some embodiments, optimized is while maintaining proper expression of the protein in the first and second tissues or cell types. In some embodiments, proper expression in the first tissue or cell type is expression. In some embodiments, proper expression in the second tissue or cell type is low or no expression. In some embodiments, optimization is while maintaining inhibition of protein expression in the absence of the trigger RNA. In some embodiments, optimization is while maintaining inhibition of protein expression in the second tissue or cell type. In some embodiments, optimization is while maintaining expression of the protein in the presence of the trigger RNA. In some embodiments, optimization is while maintaining expression of the protein in the first tissue or cell type.

In some embodiments, the RNA comprises an unhybridized region between a first and second part of the 3′ hybridized region. In some embodiments, the RNA comprises an unhybridized region between a first and second part of the 5′ hybridized region. In some embodiments, the unhybridized region is a bulge. In some embodiments, the RNA comprises an unhybridized region between a first and second part of the region reverse complementary to the 3′ hybridized region. In some embodiments, the RNA comprises an unhybridized region between a first and second part of the region reverse complementary to the 5′ hybridized region. In some embodiments, the RNA comprises an unhybridized region within both hybridized regions. In some embodiments, the unhybridized region is a bulge. In some embodiments, the unhybridized region comprises at least 1, 2, 3, 4, 5, 6, 7, 8, 9, 10, 12, 15, 17, or 20 nucleotides. Each possibility represents a separate embodiment of the invention. In some embodiments, the unhybridized region comprises at most 4, 5, 6, 7, 8, 9, 10, 12, 15, 17, 20, 25, 30, 35, 40, 45 or 50 nucleotides. Each possibility represents a separate embodiment of the invention. In some embodiments, the unhybridized region comprises at most 4 nucleotides. In some embodiments, the unhybridized region comprises at most 7 nucleotides. In some embodiments, the start codon is located in the unhybridized region. In some embodiments, the start codon is located in the unhybridized region within the region reverse complementary to the 3′ hybridized region. In some embodiments, the start codon is located in the unhybridized region within the 5′ hybridized region. In some embodiments, the linker comprises the second part of the region reverse complementary. In some embodiments, the linker comprises the second part of the 5′ hybridized region. In some embodiments, the second part is the part 3′ to the start codon. In some embodiments, the linker comprises the 3′ unhybridized region.

In some embodiments, the RNA molecule of the invention is depleted of immunogenic epitopes. In some embodiments, immunogenic epitopes are epitopes recognized by the immune system. In some embodiments, recognized by the immune system is recognized by T cells. In some embodiments, immunogenic is immunogenic to a mammal. In some embodiments, the mammal is a human. In some embodiments, an epitope algorithm is used to deplete the RNA molecule.

In some embodiments, the RNA of the invention comprises or consists from 5′ to 3′ of the reverse complement to the selected region; the loop region; the region reverse complementary to the 3′ hybridized region; and the obtained coding sequence. In some embodiment, the RNA of the invention further comprises a spacer region 5′ to the reverse complement to the selected region. In some embodiment, the RNA of the invention further comprises an additional region 3′ to the coding sequence. In some embodiments, the additional region comprises 3′ untranslated region (UTR). In some embodiments, the 3′ UTR is the endogenous 3′ UTR of the coding sequence. In some embodiments, the additional region comprises a spacer region.

In some embodiments, the method further comprises confirming the RNA molecule of the invention produces the protein in the first tissue or cell type. In some embodiments, the method further comprises confirming the RNA molecule of the invention lowly produces the protein in the second tissue or cell type. In some embodiments, lowly produces is does not produce. In some embodiments, the confirming comprises applying a trained regressor algorithm to predict protein production. In some embodiments, the algorithm is trained on sequences and known protein expression.

In some embodiments, the RNA molecule of the invention is a vector RNA molecule. A vector nucleic acid sequence generally contains at least an origin of replication for propagation in a cell and optionally additional elements, such as a heterologous polynucleotide sequence, expression control element (e.g., repressor, enhancer), selectable marker (e.g., antibiotic resistance), and/or poly-Adenine sequence.

By another aspect, there is provided an RNA molecule produced by a method of the invention.

By another aspect, there is provided an RNA molecule produced by a computer program product of the invention.

By another aspect, there is provided a cell comprising an RNA molecule of the invention.

By another aspect, there is provided a composition comprising an RNA molecule of the invention.

By another aspect, there is provided a composition comprising a cell of the invention.

By another aspect, there is provided a method of producing a cell of the invention, the method comprising producing an RNA molecule by a method of the invention and contacting a cell with the produced RNA molecule, thereby producing a cell of the invention.

By another aspect, there is provided a method of producing a composition of the invention, the method comprising producing a cell of the invention and combining the cell with a pharmaceutically acceptable carrier, excipient or adjuvant.

In some embodiments, the cell is a eukaryotic cell. In some embodiments, the cell expresses the protein of interest. In some embodiments, the cell is a cell of the first tissue or cell type. In some embodiments, a cell is a population of cells. In some embodiments, the population of cells comprises cells of the first tissue or cell type. In some embodiments, the population of cells comprises cells of the second tissue or cell type. In some embodiments, the population of cells comprises cells of the first tissue or cell type and cells of the second tissue or cell type. In some embodiments, cells of the first tissue or cell type and cells of the second tissue or cell type both comprise the RNA molecule of the invention. In some embodiments, cells of the first tissue or cell type express the protein of interest at higher levels than cells of the second tissue or cell type. In some embodiments, a higher level is at least 3, 4, 5, 6, 7, 8, 9, 10, 11, 12, 13, 14, or 15 times higher. Each possibility represents a separate embodiment of the invention. In some embodiments, a higher level is at least 3 times higher. In some embodiments, a higher level is at least 5 times higher. In some embodiments, a higher level is at least 6 times higher. In some embodiments, a higher level is at least 9 times higher. In some embodiments, a higher level is at least 10 times higher. In some embodiments, a higher level is at least 13 times higher.

In some embodiments, the composition is a therapeutic composition. In some embodiments, the composition is a diagnostic composition. In some embodiments, the composition comprises a therapeutically effective carrier, excipient or adjuvant. In some embodiments, the composition is formulated for systemic administration. In some embodiments, the composition is formulated for administration to a eukaryote. In some embodiments, the carrier, excipient or adjuvant is formulated for administration to a eukaryote. In some embodiments, the eukaryote is a mammal. In some embodiments, the mammal is a human. In some embodiments, contact comprises expressing the synthetic RNA in the cell.

By another aspect, there is provided a computer program product comprising a non-transitory computer-readable storage medium having program code embodied thereon, the program code executable by at least one hardware processor to perform a method of the invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

By another aspect, there is provided a method of producing expression of a protein of interest in a first tissue or cell type, the method comprising introducing a synthetic RNA molecule of the invention into the first tissue of cell type, thereby producing the protein of interest.

In some embodiments, the first tissue or cell type is a eukaryotic tissue or cell type. In some embodiments, the first cell type is a yeast cell. In some embodiments, the introducing further comprises introducing the synthetic RNA molecule of the invention into a second tissue or cell type. In some embodiments, the method further comprises introducing the synthetic RNA molecule of the invention into a second tissue or cell type. In some embodiments, the second tissue or cell type is different than the first tissue or cell type. In some embodiments, the introducing into the second tissue or cell type does not produce expression of the protein of interest in the second tissue or cell type. In some embodiments, the introducing into the second tissue or cell type produces low expression of the protein of interest in the second tissue or cell type. In some embodiments, the second tissue or cell type is a eukaryotic tissue or cell type. In some embodiments, the second cell type is a yeast cell.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Toehold Switch Overview

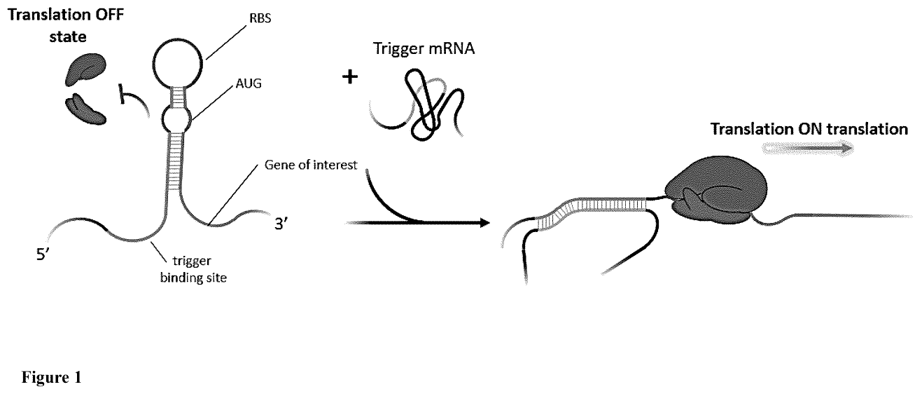

FIG. 1 depicts the concept of a toehold switch in its most basic form. The untranslated region folds into a hairpin structure and translation initiation is repressed. If a specific RNA molecule is present (the trigger mRNA), it binds the exposed 5′ area just before the beginning of the stem (trigger binding site) and then hybridizes to the strand region that is part of the stem. This hybridization opens the structure and makes available the ribosome binding and translational start site (AUG codon). The ribosome can then bind the mRNA and initiate translation of the protein of interest.

The dynamic range of the Toehold switch refers to the difference in expression achieved in a cell that expresses the trigger mRNA and a cell that does not. Wang et al., 2019, “A novel toehold switch for microRNA detection in mammalian cells”, ACS Synth. Biol. 2019, 8, 5, 1079-1088, herein incorporated by reference in its entirety, teaches the use of a toehold switch in mammalian cells for detection of a target mRNA. The toehold drives the expression of green fluorescent protein; however, the dynamic range was only 2-fold between miRNA expressing cells and non-expressing cells. This was caused both by high background expression in off target cells and limited upregulation in the target cells.

To increase the dynamic range several aspects are taken into account. First, there should be no off-target expression while maximizing on-target expression as much as possible. Another aspect is finding the perfect switch (trigger mRNA), which best distinguishes our target tissue or cell type from other tissues or cell types. Of even more importance is the efficiency of the switch, which could be affected by misfolding, ribosome affinity, locked off state and many more considerations. The Algorithm developed can be divided into two parts: 1) Finding the best trigger; and 2) Toehold switch design.

### Example 2: Finding the Best Trigger

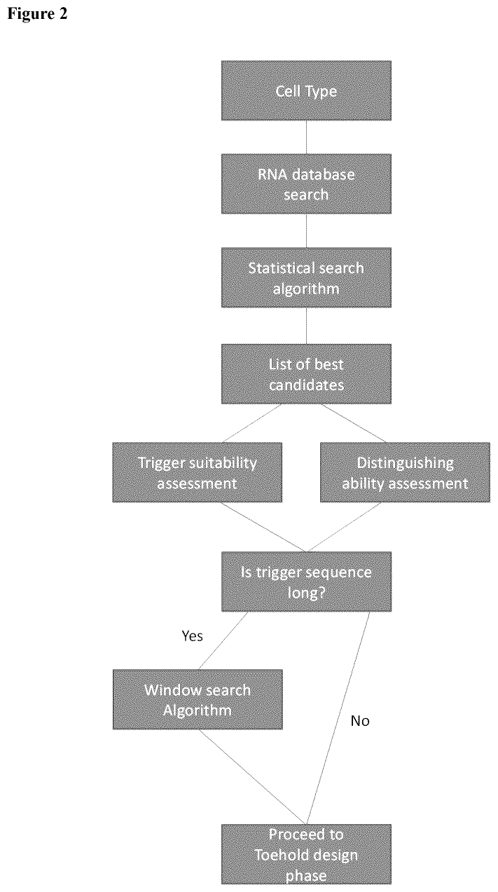

The trigger selection method is diagramed in FIG. 2. Firstly, the trigger space is expanded from miRNAs to any type of RNA molecules that fit the users' needs. This gives an increased flexibility in the process of the switch design and allows for more degrees of freedom. The algorithm finds the specific optimal RNA molecule for the relevant treated cells and the optimal sequence window within that molecule.

The best RNA molecule candidates are selected by a statistical search algorithm. The algorithm uses big data sources for creating a list of potential RNA molecules and ranking them according to distinguishing ability and trigger suitability. Distinguishing ability is based on the existence and abundance within different tissues/cell types. The best candidates are expressed in the targeted cells at such a level that activates the toehold switch but in relative low levels in the other non-targeted cells. The algorithm is based on a statistical test performed on the gene expression levels using a threshold defined by biological considerations. Another ranking parameter is the trigger suitability which derives from several characteristics such as concentration, GC content, the existence of an unfolded segment that could hybridize to the toehold and more.

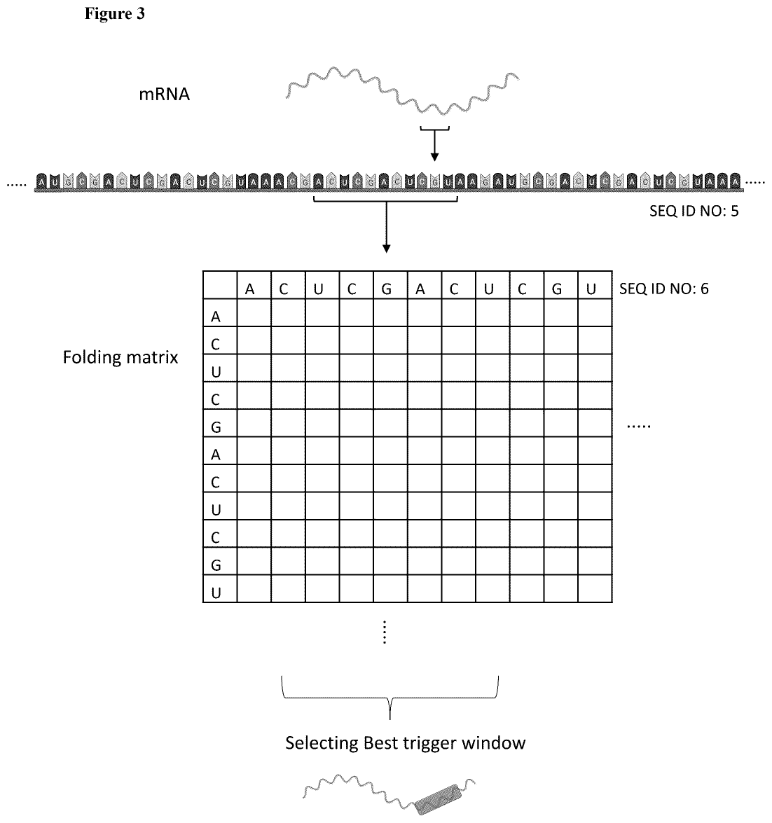

If the RNA molecule is characterized by a long sequence, e.g., an mRNA, a short sequence within it is selected as the trigger by a window search algorithm (FIG. 3). The window search algorithm is based on a folding matrix which contains the probability of each nucleotide connecting with another nucleotide in the sequence. Using this folding matrix, the complex secondary structure and ensemble options are predicted. The optimal window is a short sequence that is open and stable within the structure of the RNA molecule, so as to allow interaction with the toehold switch molecule. The diagonal of the matrix represents a sequence of consecutive nucleotides that are not bonded to other nucleotides, so a search on the diagonal is conducted for finding the most probable open window. To avoid spatial disturbances, probability and entropy traits of the nucleotides that are adjacent to the selected window are also examined.

The algorithm takes the kinetics of the system assuming non-equilibrium and by scans with a nested sliding window algorithm that makes use of a typical window size that is roughly the size of two ribosomes (˜60 nucleotides). The outer window's size is the bio-physical assumed refolding section size (section that is unfolded by a scanning ribosome). Within each such outer window, the expected hybridization probability and fold energy of every inner subsegment (inner windows) is measured. Each subsegment is considered by shifting over one nucleotide at a time. Finally, for every subsegment the contributions from all outer windows which contain the subsegment are combined to produce a score for the subsegment. Then the inner window scores are combined to produce a total score for the outer window, and the best candidates (e.g., outer windows with the highest scores) are then scanned for interactions with other endogenous RNAs in the target cell type. This optimization is done by taking all RNAs and their relative expression and finding the probable inter-molecule hybridization interference with the trigger mRNA generally and specifically with the trigger mRNA binding segment. The selection of the final trigger segment is combined with the selection of the best endogenous RNA to server as a trigger, combining high expression in the cell type of interest and also having a good trigger segment that is “orthogonal” to other RNAs in that cell.

### Example 3: Toehold Switch Design

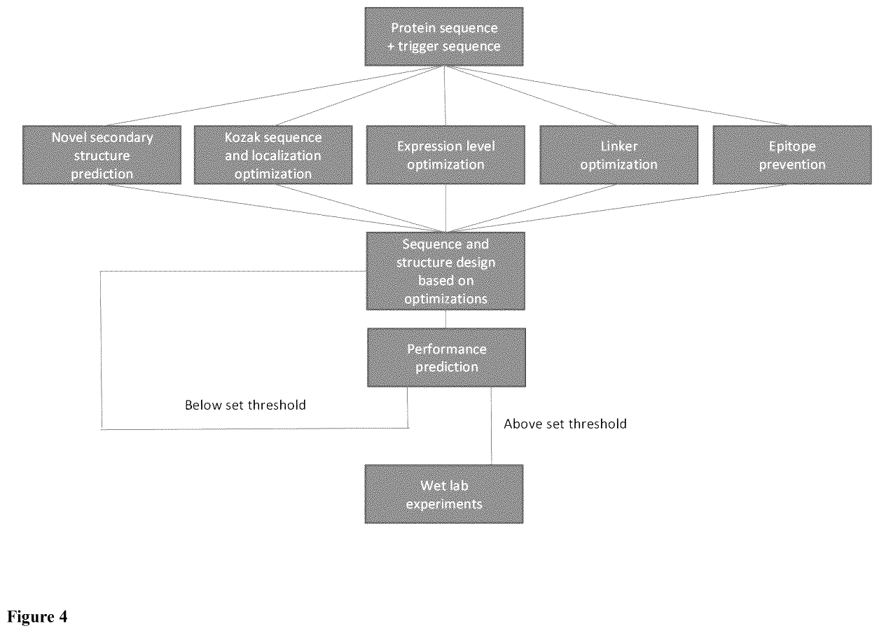

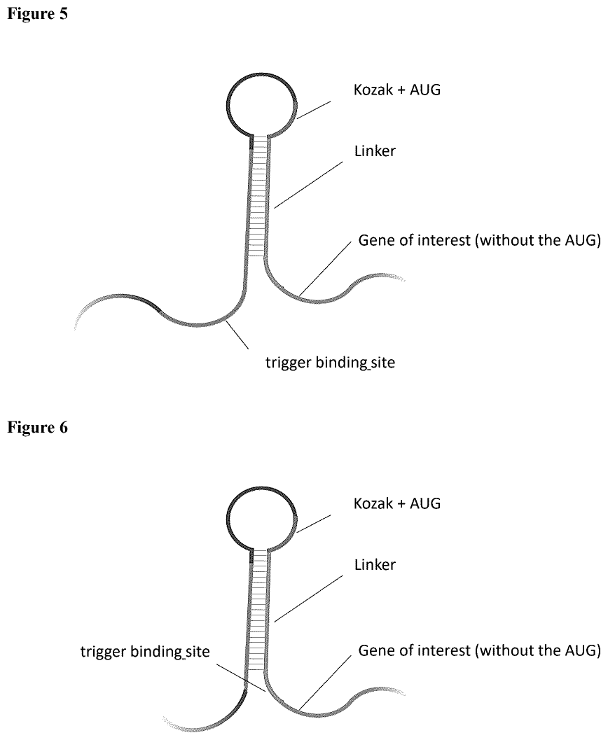

The trigger selection method is diagramed in FIG. 4. The model-based algorithm for the optimization of the process of switch design is based on optimizing structural and thermodynamical characteristics such as stem size, loop size, folding energies of the different states and hybridization energy of the trigger and the trigger binding site. The general toehold switch structure of the invention is shown in FIG. 5. The computational model takes those features as well as published data related to switch efficiency into account while designing a new switch. These key features range from low resolution features such as minimum folding energy (MFE) and GC content, to medium resolution features by looking at a partial segment of the sequence, to high resolution features such as position specific nucleotide presence. As can be seen in FIG. 5, the eukaryotic toehold of the invention places the AUG start codon not in its conventional position at the start of the gene of interest, but rather within the toehold structure. This provides for greater transcriptional control by the toehold.

Moreover, several challenges in design led to the following novel techniques:
1. Prediction of RNA secondary structure of the toehold switch (including the gene of interest) isn't reliable enough by using common tools, due to the long length of the sequence. Therefore, a new approach was developed. To increase the reliability of the prediction, the problem was converted into linear programming by using a sliding window algorithm. The algorithm weighs all predictions of fixed or different sized windows of the sequence to predict the most probable secondary structure. This approach also helps prevent unwanted pseudoknots.
2. In most studies, the access to the Kozak sequence is prevented by locating it within the hairpin. This algorithm optimizes the Kozak sequence localization in order to ensure access is prevented in the OFF state (e.g., off-target cells).
3. In most studies, the trigger binding site in the switch is positioned in the beginning of the 5′ of the switch, however, it has been determined that trigger binding sites in the 3′ side of the stem loop are also viable and enable a shorter unhybridized region at the 5′ end (FIG. 6). This reduces leakage in eukaryotes, owing to the effect of eukaryotic helicase complexes that open mRNA secondary structures.
4. The toehold switch structure is affected by the sequence of the gene of interest. The algorithm adapts the toehold switch sequence to the gene of interest. This is done according to characteristics of the gene of interest such as codon usage bias, typical decoding rate (TDR) and more. These methods also increase expression levels in the ON state (e.g., target cells). Optimization of the best structures was performed by codon optimization to avoid unwanted interactions. For example, hybridization of the trigger binding site with the gene of interest was avoided and at the same time the expected translation rate was optimized by modeling optimized codon bias sequences for translation.
5. In most studies, the segment in the trigger gene to be used as a trigger sequence is chosen as a predicted unhybridized/unbound segment in the structure of that gene. The algorithm of the invention also takes into consideration the avoidance of unwanted hybridization with the gene of interest itself when selecting the endogenous gene to be selected as a trigger. Thus, this trigger molecule must be highly expressed but also be optimized to hybridize well to open the switch but not co-fold with the gene of interest.
6. In most studies, the structures that are being predicted and used for optimization are generated assuming equilibrium and using ensembles and partition functions according to thermodynamics of equilibrium. However, the algorithm of the invention takes into consideration the kinetics of the system during the optimization process. This produces more accurate modeling due to RNA being less stable than DNA and mRNA being actively translated both for the gene of interest in the switch and the triggering mRNA. Specifically, that issue of kinetics is the reason for the robust algorithm with a moving window during the trigger selection which finds endogenous gene segments appropriate for binding the toehold switch.
7. In most studies, the optimization algorithm maximizes the on/off dynamic ratio, herein the algorithm of the invention also optimizes having a high on level and not only the on/off ratio.
8. The linker, the sequence between the start codon (located within the toehold structure) and the rest of the gene of interest, has an effect on the expression of the gene of interest. Therefore, the algorithm optimizes the length of the linker and its sequence in order to produce the shortest possible linker that will not affect the toehold switch activity which distinguishes the OFF and ON states.
9. Preventing T cell epitope (immunogenic epitopes recognized by T cells) entrance in all algorithm stages is highly important.

The performance of the designed switch is predicted using a regressor which uses key features and is trained on a large database. If the performance is above a certain set threshold, the sequence moves forward to wet lab validation. If not, the design phase is repeated.

### Example 4

In order to evaluate the abilities of the tools produced a focus on cancerous conditions was adopted. Using current approaches, cancer cells are hard to target. Therefore, an algorithm was developed that designs toehold switches that are activated in cancer cells but not in healthy cells. This is achieved by limiting the trigger to genes regions containing cancer specific mutations. The mutations are the key that distinguishes between healthy and the cancer cells.

The first step of the algorithm is to find a list of optional trigger sequences which meet the following conditions:
1. There is a 30-36 base-pair length window with mutations that exist only in the cancer genome but not in the healthy genome.
2. The mutation/s causes significant variance between the cancer genome and the healthy genome within the selected window.
3. The mutations appear in the mRNA.
4. The mutations are common/shared in a large group of patients.

The second step of the algorithm is to design Toehold switches optimized to each of the trigger sequences in the optional list. The third and last step is to rate the optional trigger sequences according to their folding performance, the more open the structure the better, and according to the interaction energy of the toehold and its corresponding trigger sequence, the higher the energy the better (the maximum energy difference producing the maximum difference in expression in cancer and healthy cells).

### Example 5: Proof of Concept Confirmation

The entire process of improving the dynamic range of toehold switches in eukaryotes and mammalian cells and the creation of computational models for the efficient design of the switches is paired with wet lab experiments to confirm effectiveness. By working in several cycles, a model is designed, switches are built according to the model, built switches are tested and conclusion are drawn for the next cycle. The results obtained by using miRNA-155 as a trigger for a toehold switch that gates the expression of GFP (see Wang et al.) were reproduced. The plasmids that were originally used in this experiment were obtained and co-express along with the trigger miRNA-155 in HEK293T cells (which do not express miR-155 endogenously) to simulate an on-state. Similarly, the toehold-GFP plasmid was expressed alone without expressing the trigger to simulate the off-state. This was treated as the baseline cycle.

For the next cycle, an optimized sequence for the miR-155-toehold-GFP is outputted by the algorithm, synthesized, and tested in a same manner. Future cycles include different mRNA triggers for the toehold design and are tested in a similar manner. Additionally, the synthesized constructs are testing in mammalian cells, e.g., yeast.

### Example 6: Proof of Concept Confirmation in Yeast with the Kozak Sequence in the Toehold

Yeast cells were made to express mCherry as the trigger molecule as a proof of concept. four distinct toehold molecules were tested (SEQ ID NO: 1-4). Two different trigger sequences were identified within an mCherry sequence that was codon optimized for expression in yeast. The first was incorporated into the toehold of SEQ ID NO: 1 and 3. The second was incorporated into the toehold of SEQ ID NO: 2 and 4.

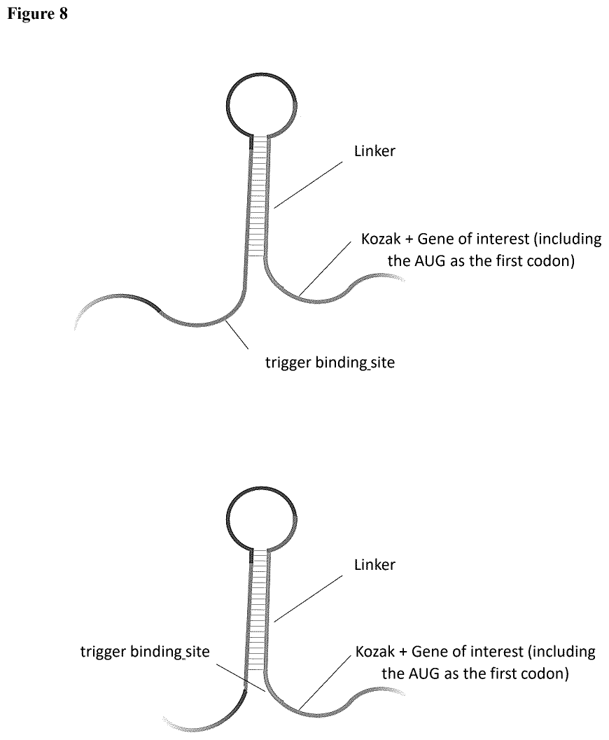

The position of the Kozak sequence was also tested. The Kozak sequence and ATG was either left directly 5′ adjacent to the gene of interest (GFP) (FIG. 8) or it was placed within the loop region of the stem-loop of the toe hold (FIG. 5). The 3′ stem sequence thus acts as a linker linking the ATG to the gene of interest (GFP). The Kozak and ATG were left adjacent to GFP in SEQ ID NO: 1-2 and was placed within the loop in SEQ ID NO: 3-4.

The experimental procedure was conducted as follows:
1. Construct Acquisition: The necessary constructs were procured by Twist Bioscience. Subsequently, a gap repair technique was employed for cloning purposes.
2. Yeast Cultivation: The yeast strains containing the constructed plasmids were cultivated on selective media suited for the respective plasmid. This ensured the retention and proper expression of the genetic material.
3. For the trigger candidates, an additional step was incorporated into the experimental process. Following the initial cloning, a second round of cloning was undertaken, utilizing an alternative selection media. This supplementary round of cloning aimed to fine-tune the selection process and ensure the successful incorporation and expression of the mCherry trigger candidates within the yeast host.
4. Starter Cultures: Overnight cultures were prepared from the cultivated yeast strains. These starter cultures served as the foundation for subsequent steps.
5. Logarithmic Growth Phase: The overnight starter cultures were diluted appropriately to achieve a logarithmic growth phase. This ensured a consistent and actively growing yeast population for the experimental phase.
6. Fluorescence and OD Measurements: In the OD range of 0.7 to 1, measurements were taken for both fluorescence and optical density (OD). These measurements allowed for monitoring both the expression of the fluorescent protein and the yeast growth dynamics simultaneously.
7. Normalization: To account for variations in yeast growth between samples, the obtained fluorescence measurements were normalized by the corresponding OD measurements. This normalization step ensured that the observed fluorescence was accurately representative of the expression levels relative to the growth of yeast cells.

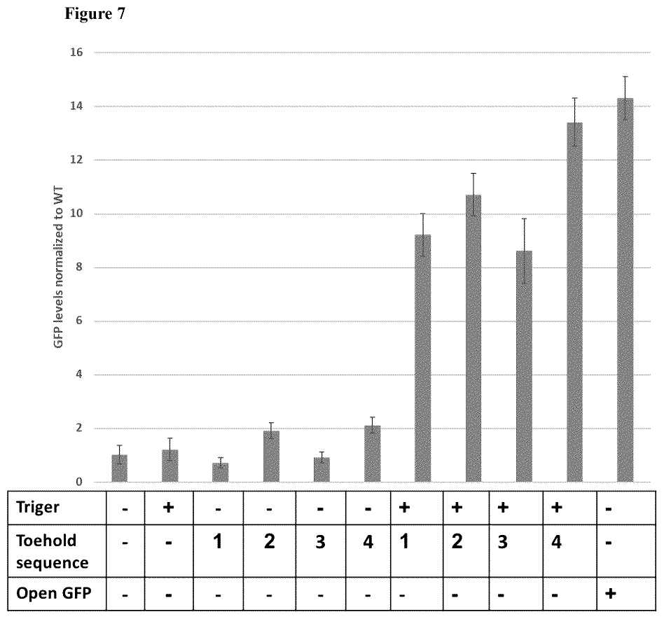

The toehold molecules as well as mCherry were expressed in yeast cells as shown in FIG. 7 and GFP fluorescence was measured. Baseline GFP fluorescence was measured in the absence of any toehold molecule (with or without mCherry). All molecules showed background levels of GFP expression indicating that the toehold was locking down expression. Surprisingly, even when the Kozak and start codon were after the toehold structure, translation was shut off in the absence of the trigger. When codon optimized mCherry was present, the trigger sequence was hybridized and expression of GFP occurred in all molecules. Although, positioning of the Kozak and ATG either in the loop or after the stem produced high levels of expression, the highest level was actually produced when the Kozak/ATG was positioned in the loop (SEQ ID NO: 4). Indeed, this construct was able to produce GFP levels that were comparable to expressing an open GFP plasmid in the cells, whereas the other constructs could not. Importantly, the dynamic range produced between the trigger negative cells and the trigger positive cells was well above any previous toeholds expressed in eukaryotic cells. The dynamic range can be simply determined by calculating the ratio of expression in the trigger positive cells to the expression in trigger negative cells. SEQ ID NO: 1 produced a ratio of 13.14, SEQ ID NO: 2 produced a ratio of 5.56, SEQ ID NO: 3 produced a ratio of 9.56 and SEQ ID NO: 4 produced a ratio of 6.38. By dynamic range, SEQ ID NO: 1 with the Kozak and ATG after the toehold, was the best synthetic RNA.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of designing a synthetic RNA molecule, the method comprising:
a. obtaining a coding sequence encoding a protein of interest;
b. obtaining a sequence of an RNA expressed in a first tissue or cell type and not expressed or lowly expressed in a second tissue or cell type;
c. selecting a region within said sequence of an RNA; and
d. producing a sequence of a synthetic RNA molecule comprising
i. from 5′ to 3′:
1. a reverse complement to said selected region comprising a 5′ unhybridized region and a 3′ hybridized region;
2. a loop region that does not hybridize to any of (1), (2) and (3);
3. a region reverse complementary to said 3′ hybridized region of (1); and
4. said obtained coding sequence encoding a protein of interest;
wherein a ribosome binding site (RBS) is located within said loop region or between region (3) and region (4); or
ii. from 3′ to 5′:
1. said obtained coding sequence encoding a protein of interest;
2. a reverse complement to said selected region comprising a 3′ unhybridized region and a 5′ hybridized region;
3. a loop region does not hybridize to (2), (3) and (4) and
4. a region reverse complementary to said 5′ hybridized region of (2);
wherein a RBS is located within said loop region or between regions (1) and (2);
thereby designing a synthetic RNA molecule.

2. The method of claim 1, wherein at least one of:
a. said synthetic RNA molecule produces said protein of interest in said first tissue or cell type and does not produce or lowly produces said protein of interest in said second tissue or cell type;
b. said coding sequence encoding a protein of interest is codon optimized for expression in said first tissue or cell type; and
c. said RNA is a messenger RNA (mRNA).

3. (canceled)

4. The method of claim 1, wherein said RNA expressed in a first tissue or cell type and not expressed or lowly expressed in a second tissue or cell type is not a microRNA (miRNA).

5. (canceled)

6. The method of claim 1, wherein said obtaining a sequence of an RNA comprises performing a statistical search algorithm on RNA expression data from said first tissue or cell type and said second tissue or cell type and ranking molecules based on their ability to distinguish said first tissue or cell type and said second tissue or cell type; optionally comprising selecting an RNA whose expression in said second tissue or cell type is sufficiently low as to not induce expression of said protein of interest in the presence of said synthetic RNA molecule and whose expression in said first tissue or cell type is sufficiently high as to induce expression of said protein of interest in the presence of said synthetic RNA molecule or selecting an RNA whose GC content is below a predetermined threshold.

7. (canceled)

8. (canceled)

9. The method of claim 1, wherein selecting a region comprises selecting an unfolded region.

10. The method of claim 9, wherein an unfolded region is a region with a local folding energy above a predetermined threshold or wherein said folding is determined for said sequence of an RN.

11. (canceled)

12. The method of claim 9, wherein
a. selecting an unfolded region comprises performing a window search algorithm that produces a folding matrix indicating the probability of each nucleotide of said sequence of an RNA hybridizing to another nucleotide of said sequence of an RNA and selecting a window predicted to not hybridize;
b. selecting an unfolded region comprises performing a window search algorithm that produces a folding matrix indicating the probability of each nucleotide of said sequence of an RNA hybridizing to another nucleotide of said sequence of an RNA and selecting a window predicted not to hybridize, wherein said another nucleotide of said sequence comprises the sequence of the gene of interest; or
c. selecting an unfolded region comprises performing a window search algorithm that produces a folding matrix indicating the probability of each nucleotide of said sequence of an RNA hybridizing to another nucleotide of said sequence of an RNA and selecting a window predicted to not hybridize.

13. (canceled)

14. (canceled)

15. The method of claim 1, wherein said region is between 20 and 100 nucleotides.

16. The method of claim 12, wherein said window is an outer window and wherein each outer window is subdivided into inner windows consisting of a portion that is less than 100% of said outer window and wherein said producing a folding matrix is producing a folding matrix for each inner window, optionally wherein said selecting an unfolded region further comprises summing the folding energies for each inner window across all outer windows to produce an inner window folding value, summing the inner window folding values of all inner windows in each outer window to produce an outer window folding value and selecting an outer window with a folding value beyond a predetermined threshold.

17. (canceled)

18. The method of claim 1, further comprising confirming formation of a stem-loop structure comprising said 3′ hybridized region, said loop region and said region reverse complementary to said 3′ hybridized region or said 5′ hybridized region, said loop region and said region reverse complementary to said 5′ hybridized region, optionally wherein said confirming comprises performing a sliding window RNA folding prediction algorithm across the full sequence of the synthetic RNA and selecting the most probable secondary structure.

19. (canceled)

20. The method of claim 1, wherein said first tissue or cell type is a eukaryotic tissue or cell type and said RBS is a Kozak sequence.

21. (canceled)

22. The method of claim 1, wherein a start codon is located in said loop region and is 3′ to said RBS, optionally wherein said synthetic RNA sequence between said start codon and said obtained coding sequence encoding a protein of interest is codon optimized to match a codon usage of said coding sequence encoding a protein of interest, further optionally wherein said codon optimization comprises codon usage bias (CUB) optimization or typical decoding rate (TDR) optimization.

23. (canceled)

24. (canceled)

25. (canceled)

26. The method of claim 1, wherein a start codon of said coding sequence encoding a protein of interest is separated from the rest of said coding sequence by a linker and wherein at least one of:
a. said start codon is downstream of said RBS and said linker is within said region reverse complementary to said 3′ hybridized region or comprises said 5′ hybridized region;
b. said method comprises optimizing said linker to be as short as possible while expression of said protein of interest is still inhibited in the absence of said RNA expressed in a first tissue or cell type and not expressed or lowly expressed in a second tissue or cell type; and
c. said method comprises codon optimizing said linker to match a codon usage of said coding sequence encoding a protein of interest.

27. (canceled)

28. (canceled)

29. The method of claim 1, wherein said synthetic RNA molecule further comprises an unhybridized region between a first and second part of said 3′ hybridized region, an unhybridized region between a first and second part of said region reverse complementary to said 3′ hybridized region or both; or an unhybridized region between a first and second part of said 5′ hybridized region, an unhybridized region between a first and second part of said region reverse complementary to said 5′ hybridized region or both.

30. The method of claim 29, wherein said start codon is located in said unhybridized region between a first and second part of said region reverse complementary to said 3′ hybridized region or in said unhybridized region between a first and second part of said 5′ hybridized region, optionally wherein said linker comprises said second part of said region reverse complementary to said 3′ hybridized region which is 3′ to said start codon or said second part of said 5′ hybridized region with is 3′ to said start codon.

31. (canceled)

32. The method of claim 1, further comprising confirming said synthetic RNA molecule produces said protein of interest in said first tissue or cell type and does not produce or lowly produces said protein of interest in said second tissue or cell type, optionally comprising applying a trained regressor algorithm to predict protein of interest production.

33. (canceled)

34. The method of claim 1, wherein said loop region is not a bulge region.

35. A synthetic RNA molecule produced by the method of claim 1.

36. A computer program product comprising a non-transitory computer-readable storage medium having program code embodied thereon, the program code executable by at least one hardware processor to perform the method of claim 1.

37. A method of producing expression of a protein of interest in a first eukaryotic tissue or cell type and not producing expression or lowly producing expression in a second eukaryotic tissue or cell type, the method comprising introducing the synthetic RNA molecule of claim 35 into said first eukaryotic tissue or cell type and said second eukaryotic tissue or cell type, thereby producing expression of a protein of interest in a first eukaryotic tissue or cell type and not producing or lowly producing expression of said protein of interest in said second eukaryotic tissue or cell type.

38. (canceled)


SEQUENCE LISTING (6 sequences)

SEQ ID NO: 1 - RNA, 1781 nt, synthetic construct
  source 1..1781: mol_type = other RNA
tgggtaacgccagggttttcccagtcacgacgttgtaaaacgacggccagtgagcgcgcgtaatacgactcactatagggcgaattgggtaccgggccccccctcgaggtcgacggtatcgatatatatggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaagaaaaattcttcttcgactatgctggaggcagagatgatcgagccggtagttaactatatatagctaaattggttccatcaccttcttttctggtgtcgctccttctagtgctatttctggcttttcctattcttttctttccatttttctttctctctttctaatatataaattctcttgcattttctatttttctctctatctattctacttgtttattcccttcaaggtttttttttaaggagtacttgtttttagaatatacggtcaacgaactataattaactaaacacttaaaaagggtttggttttgtaggttgtctttacttctgtattataaatctaacaacacaaatttataatacagaaaccaaaatgagaaaaggcgaagaattattcactggagttgtcccaattcttgttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataataaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggcggtggagctccagcttttgttccctttagtgagggttaattgcgcgcttggcgtaatcatggtcatagctgtttcctgtgtgaaattg

SEQ ID NO: 2 - RNA, 1781 nt, synthetic construct
  source 1..1781: mol_type = other RNA
tgggtaacgccagggttttcccagtcacgacgttgtaaaacgacggccagtgagcgcgcgtaatacgactcactatagggcgaattgggtaccgggccccccctcgaggtcgacggtatcgatatatatggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaagaaaaattcttcttcgactatgctggaggcagagatgatcgagccggtagttaactatatatagctaaattggttccatcaccttcttttctggtgtcgctccttctagtgctatttctggcttttcctattcttttctttccatttttctttctctctttctaatatataaattctcttgcattttctatttttctctctatctattctacttgtttattcccttcaaggtttttttttaaggagtacttgtttttagaatatacggtcaacgaactataattaactaaacacttaaaaagggtttggttttgtaggttgtctttacttctgtattataaatctaacaacacaaatttataatacagaaaccaaaatgaggaagggcgaagaattattcactggagttgtcccaattcttgttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataataaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggcggtggagctccagcttttgttccctttagtgagggttaattgcgcgcttggcgtaatcatggtcatagctgtttcctgtgtgaaattg

SEQ ID NO: 3 - RNA, 1793 nt, synthetic construct
  source 1..1793: mol_type = other RNA
tgggtaacgccagggttttcccagtcacgacgttgtaaaacgacggccagtgagcgcgcgtaatacgactcactatagggcgaattgggtaccgggccccccctcgaggtcgacggtatcgatatatatggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaagaaaaattcttcttcgactatgctggaggcagagatgatcgagccggtagttaactatatatagctaaattggttccatcaccttcttttctggtgtcgctccttctagtgctatttctggcttttcctattcttttctttccatttttctttctctctttctaatatataaattctcttgcattttctatttttctctctatctattctacttgtttattcccttcaaggtttttttttaaggagtacttgtttttagaatatacggtcaacgaactataattaactaaacacttaaaaagggtttggttttgtaggttgtttttacttttgtgtcgtagaaaccaaaatgtctttctgtggcacgaaaaacctggctgctgctcagaaacgcaaaggagaagaacttttcactggagttgtcccaattcttgttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataataaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggcggtggagctccagcttttgttccctttagtgagggttaattgcgcgcttggcgtaatcatggtcatagctgtttcctgtgtgaaattg

SEQ ID NO: 4 - RNA, 1793 nt, synthetic construct
  source 1..1793: mol_type = other RNA
tgggtaacgccagggttttcccagtcacgacgttgtaaaacgacggccagtgagcgcgcgtaatacgactcactatagggcgaattgggtaccgggccccccctcgaggtcgacggtatcgatatatatggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaagaaaaattcttcttcgactatgctggaggcagagatgatcgagccggtagttaactatatatagctaaattggttccatcaccttcttttctggtgtcgctccttctagtgctatttctggcttttcctattcttttctttccatttttctttctctctttctaatatataaattctcttgcattttctatttttctctctatctattctacttgtttattcccttcaaggtttttttttaaggagtacttgtttttagaatatacggtcaacgaactataattaactaaacacttaaaaagggtttggttttgtaggttgtttttacttttgtgtcgtagaaaccaaaatgtctttctgtggcacgaaaaacctggcggcggcacagaaacgtaaaggagaagaacttttcactggagttgtcccaattcttgttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataataaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggcggtggagctccagcttttgttccctttagtgagggttaattgcgcgcttggcgtaatcatggtcatagctgtttcctgtgtgaaattg

SEQ ID NO: 5 - RNA, 54 nt, synthetic construct
  source 1..54: mol_type = other RNA
atgcgactcgactcgtaaacgactcgactcgtaagatgcgactcgactcgtaaa

SEQ ID NO: 6 - RNA, 11 nt, synthetic construct
  source 1..11: mol_type = other RNA
actcgactcgt